(19) United States
(12) Patent Application Publication
(10) Pub. No.: US 2026/0041516 A1
(43) Pub. Date: Feb. 12, 2026

(54) SECURING ELEMENT IN A MEDICAL CARRIER SYSTEM

(71) Applicant: Mavig GmbH, Muenchen (DE)

(72) Inventors: Marc HOFFSTETTER, Muenchen (DE); Simon SCHMITT, Ehrenberg (DE)

(73) Assignee: Mavig GmbH, Muenchen (DE)

(21) Appl. No.: 19/294,859

(22) Filed: Aug. 8, 2025

(30) Foreign Application Priority Data
Aug. 8, 2024 (DE) 102024122711.7

(51) Int. Cl. A61B 50/28; F16B 7/04

(52) U.S. Cl. CPC A61B 50/28; F16B 7/0406

(57) ABSTRACT

The invention relates, among other things, to a securing element for attachment to a cylindrical connecting element with a groove extending radially around the cylindrical connecting element, wherein the securing element comprises: two parts, wherein the two parts each comprise a bulge; and an elastic element which is adapted to press the two parts together into a compressed state; wherein the respective bulge is adapted to fit into the groove of the cylindrical connecting element and, in the compressed state, to engage in the groove of the cylindrical connecting element in such a manner that the securing element cannot be stripped off the connecting element.

The disclosure relates to a securing element for attachment to a cylindrical connecting element with a groove, a system comprising the securing element and the cylindrical connecting element, and a use of the securing element.

Securing elements are generally attached to connecting elements in order to preferably attach arms to the connecting element. This creates an arm system. With known securing elements, securing elements can only be attached with considerable effort. Several hands are required because known systems are complex. Furthermore, tools are required, and assembly/disassembly is time-consuming.

The aforementioned disadvantages of the prior art are at least partially eliminated or reduced by the features of the independent claims. Dependent claims disclose preferred embodiments of the invention.

In detail, the disclosure comprises a securing element for attachment to a cylindrical connecting element with a groove extending radially around the cylindrical connecting element, wherein the securing element comprises: two parts, wherein the two parts each comprise a bulge; and an elastic element which is adapted to press the two parts together into a compressed state; wherein the respective bulge is adapted to fit into the groove of the cylindrical connecting element and, in the compressed state, to engage in the groove of the cylindrical connecting element in such a way that the securing element cannot be stripped off the connecting element.

The elastic element, which presses the two (or more) parts together, can make it easier to assemble or disassemble the securing element. For this purpose, the two parts are pressed apart (i.e., a force acts from the inside to the outside and presses the two halves of the securing element apart) or pulled apart (i.e., the two halves of the securing element are pulled apart by forces acting from the outside). The securing element may be pressed apart when the securing element is pulled onto the connecting element (e.g. by means of a chamfer on the connecting element). The parts may be pulled apart before the securing element is pulled onto the connecting element (e.g. by the user). A combination of pressing apart and pulling apart is also possible. The securing element is pulled off in an equivalent manner.

After the securing element has been pulled on, the elastic element presses the two parts back together. Either the bulges engage directly in the groove of the connecting element, or the securing element may be pushed into the correct position in which the bulges engage in the groove. The securing element is attached when the bulges engage in the groove.

The cylindrical connecting element can be a tripod pin and the groove of the cylindrical connecting element can be a recess. The elastic element can be a spring, a rubber band, an elastic band, or the like.

The cylindrical connecting element is preferably part of a holding arm system that is fixedly or movably (e.g., on rollers) attached to a ceiling, a wall, or the floor. The cylindrical connecting element preferably corresponds to an end portion of a ceiling bracket to which one or more arms of the holding arm system are attached (by means of tubes that are pulled over the connecting element). The cylindrical connecting element is preferably designed as an elongated hollow body and/or made of a substantially inflexible material.

Various embodiments may preferably comprise the following features.

Preferably, each respective bulge has a respective chamfer.

The chamfer may be an inclined surface. The inclined surface presses the two parts apart when the inclined surface meets the cylindrical connecting element. This facilitates the pulling onto the cylindrical connecting element.

All of the respective bulges may extend all the way around, i.e., continuously in the circumferential direction on the entire inner surface of the respective part.

Alternatively, the respective bulge may comprise a single (or a few) small bulges that cover only a fraction of the circumference of the cylindrical connecting element. These small bulges may still engage in the groove of the cylindrical connecting element and thus secure the securing element. The small bulges may be dot-shaped, small lines (circle segments) or the like.

Preferably, the respective bulges are aligned with a side facing the respective other part.

The side facing the respective other part may be on the inside. In this way, the bulges engage inwardly in the groove of the cylindrical connecting element when the two parts are pressed together by the elastic element.

Preferably, the securing element is adapted to be pulled at least partially over an axial end of the connecting element.

Preferably, the securing element further comprises a locking element which is adapted to fix the securing element in the compressed state.

This increases safety, as the securing element (when fully assembled and the system is in use) cannot be accidentally pulled apart and thus pulled off the cylindrical connecting element.

Preferably, the locking element is at least one screw, preferably two screws, wherein the two screws are preferably arranged symmetrically on the securing element.

The screw(s) may be screwed in and thereby fix the securing element.

The locking element may consist of a screw and a nut, wherein the screw may be guided through bores in the two parts and may be fastened with the nut. Two (or more) screws and two (or more) nuts and correspondingly two (or more) bores through the two parts are also possible.

Preferably, the securing element further comprises two rods (rod-shaped elements), wherein the two parts each comprise two bores, wherein a respective one of the two rods is adapted to be guided through a respective bore in one of the two parts.

The rods stabilize the securing element during assembly. When the rods extend through the bores, the securing element cannot tilt (in the pulled-apart state).

Preferably, at least one of the two rods is hollow and comprises a thread which is adapted to receive the screw, and each of the two rods comprises a respective locking wall which is located on an outer side of the respective one of the two rods and which is larger than a diameter of the bores of the two parts.

The locking wall may prevent the corresponding rod (for example, during screwing in of the corresponding screw) from being pulled through the corresponding one of the two parts and thus providing no hold. Preferably, the locking wall is on the outside (on the side of the one part facing away from the other part).

Preferably, the respective bulges are together adapted to extend, in the compressed state, radially around the connecting element in the groove.

Preferably, the securing element is adapted to have a central bore in the radial center in the compressed state.

A further screw may be guided through the central bore and screwed into the cylindrical connecting element. This allows the securing element to be further secured.

Preferably, the securing element further comprises: more than two parts comprising the two parts, wherein the more than two parts each comprise a bulge.

The more than two parts (including the two parts mentioned above) may be three, four, or even more parts in total. There may also be (additional) parts that do not have a bulge. The more than two parts (as in the case when only two parts are included) may partially interlock. This may stabilize the parts. Alternatively, the parts may have flat surfaces on walls that are adjacent to each other. The parts may then only be stabilized by pressing the elastic element together. There may also be parts (/pairs of parts) that interlock and other parts (/pairs of parts) that have flat surfaces on adjacent walls. In other words, the present disclosure is not limited to the number of parts. However, due to the simpler manufacture and for reasons of stability, the two-part embodiment is preferred.

The disclosure also includes a system comprising: a cylindrical connecting element with a groove extending radially around the cylindrical connecting element; and the securing element described above.

Preferably, the cylindrical connecting element has a chamfer.

The chamfer may be an inclined surface. This may facilitate the pulling of the securing element onto the cylindrical connecting element by allowing the inclined surface to push the two parts of the securing element apart when the inclined surface encounters the securing element.

Preferably, the system further comprises a tube with an inner diameter greater than an outer diameter of the connecting element and smaller than an outer diameter of the securing element.

The tube may be pulled onto the cylindrical connecting element and fixed by the securing element so that the tube can no longer be pulled off the cylindrical connecting element. A holding arm may be attached to the tube. This holding arm may hold a screen, a bracket, or the like.

The cylindrical connecting element is preferably a medical stand device that is adapted for use in an operating theater and for positioning medical equipment.

The disclosure also includes a use of a securing element according to one of the aspects disclosed herein in the assembly of a cylindrical connecting element, which is preferably adapted for use in an operating theater, wherein during assembly, the respective bulge is brought into engagement with the groove of the cylindrical connecting element and the elastic element is arranged to bring the two parts into a compressed state so that the securing element cannot be stripped off the connecting element.

A corresponding method may comprise the following steps: (optional) pulling a tube onto the cylindrical connecting element; pulling the two parts apart against the elastic force of the elastic element; pulling the securing element onto the cylindrical connecting element; bringing the two parts together (possibly by releasing them, whereby the elastic element presses the two parts together); if present, fastening the locking element.

Alternatively, a corresponding method may comprise the following steps: (optional) pulling a tube onto the cylindrical connecting element; pulling the securing element onto the cylindrical connecting element, whereby the two parts are first pressed apart and then brought together when being pulled on (preferably by pulling them on over a chamfer); if present, fastening the locking element.

The advantages described are neither limiting nor exclusive to the corresponding aspects. An aspect may have further advantages not explicitly mentioned.

The exemplary embodiments and examples disclosed herein are intended to provide features that are readily apparent from reference to the following description in conjunction with the accompanying Figures. In accordance with various embodiments, exemplary systems, methods, and devices are disclosed herein. However, it is understood that these embodiments are presented as examples and not as limitations, and it will be apparent to those who have read the present disclosure and have normal technical knowledge that various modifications may be made to the disclosed embodiments while remaining within the scope of the present disclosure.

Therefore, the present disclosure is not limited to the exemplary embodiments and applications described and illustrated herein. Furthermore, the specific order and/or hierarchy of the steps in the methods disclosed herein are merely exemplary approaches. Based on design preferences, the specific order or hierarchy of the steps of the disclosed methods or processes may be rearranged while remaining within the scope of the present disclosure. Those skilled in the art will therefore understand that the methods and techniques disclosed herein represent various steps or actions in an exemplary order, and the present disclosure is not limited to the specific order or hierarchy unless expressly stated otherwise.

It is also understood that any reference to an element here with a designation such as “first,” “second,” etc. does not generally limit the quantity or order of these elements. Rather, these designations may be used here as a practical means of distinguishing between two or more elements or instances of an element. The reference to a first and a second element therefore does not mean that only two elements can be used or that the first element must precede the second element in any way.

Various modifications of the implementations described in this disclosure are readily apparent to those skilled in the art, and the general principles defined herein can be applied to other implementations without departing from the scope of this disclosure. Therefore, the disclosure is not limited to the implementations shown herein but has the widest scope compatible with the new features and principles disclosed herein, as set forth in the claims below.

The above and other aspects and their implementations are described in more detail in the drawings, descriptions, and claims.

### FIG. 1 Shows a Schematic Representation of an Arm System According to an

embodiment.

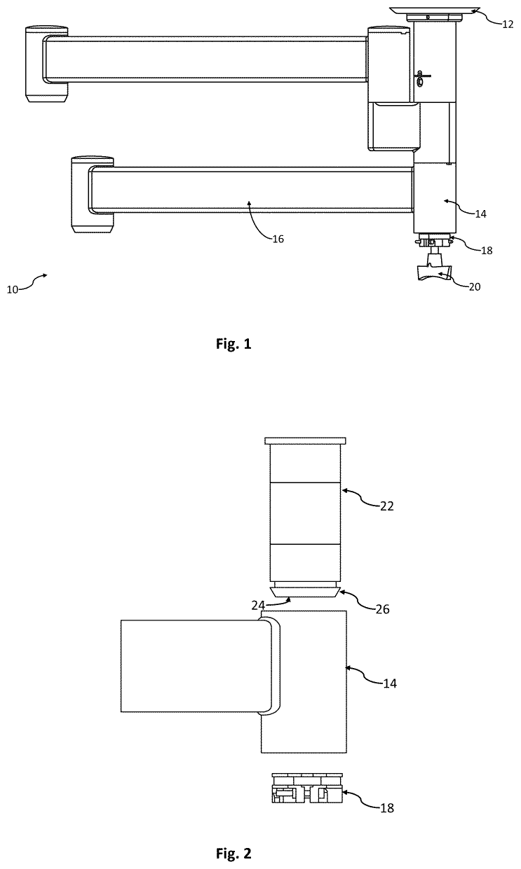

FIG. 2 shows a schematic representation of an arm system broken down into its components in perspective according to an embodiment.

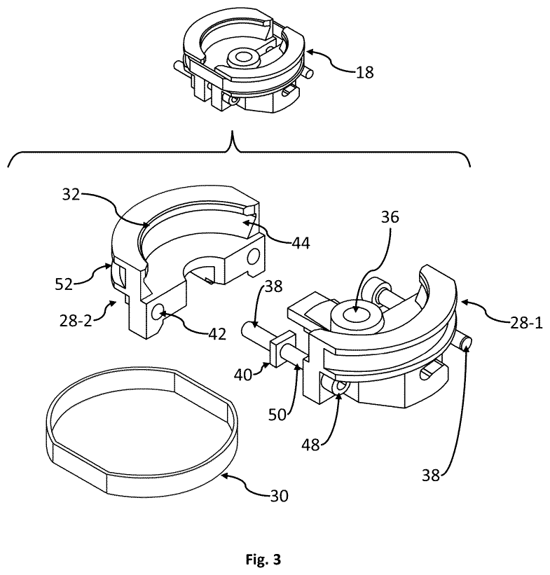

FIG. 3 shows a schematic representation of an exemplary securing element according to an embodiment.

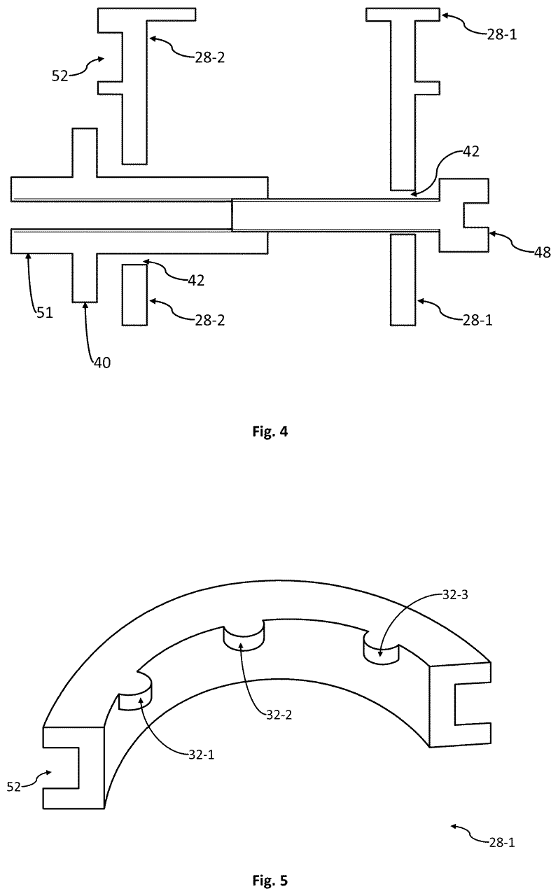

FIG. 4 shows a schematic representation of the locking elements according to an embodiment.

FIG. 5 shows a schematic representation of one of the two parts according to an embodiment.

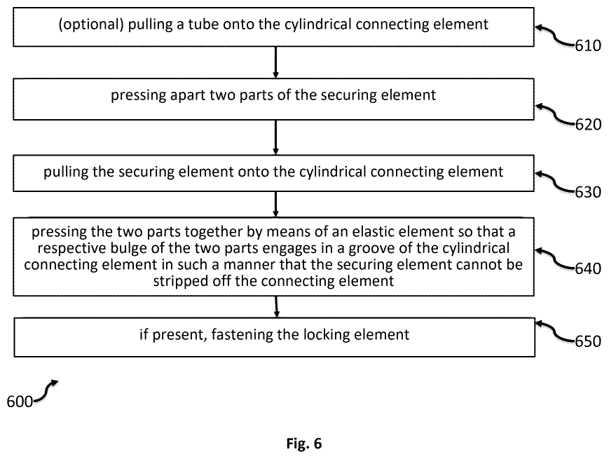

FIG. 6 shows an example method for attaching the securing element to the cylindrical connecting element according to an embodiment.

FIG. 1 shows a schematic representation of an arm system 10 according to an embodiment. The arm system 10 comprises a ceiling bracket 12, a plurality of tubes 14 with arms 16, a securing element 18, and a further screw 20. Furthermore, the arm system 10 comprises a cylindrical connecting element (in short: connecting element) onto which the tubes 14 are pulled and which is not directly visible in FIG. 1. The arm system 10 is held to a ceiling by the ceiling bracket 12. The ceiling bracket 12 can take various forms. The securing element 18 and the further screw 20 fasten the tubes 14 to the connecting element.

FIG. 2 shows a schematic representation of an arm system 10 broken down into its components in perspective according to an embodiment. The cylindrical connecting element 22 has a smaller diameter than the tube 14. This allows the tube to be pulled over the connecting element 22. The connecting element 22 can be a tripod pin. The connecting element 22 has an axial end 24 on its lower side. The axial end 24 has a groove 26. The groove 26 may be an undercut behind a chamfer. The chamfer has an inclined surface that tapers downwards (towards the axial end 24). This makes it easier to pull on a securing element 18. Furthermore, the two parts of the securing element 18 may be pressed apart by the chamfer and snap into place behind the chamfer in the groove 26, whereby the snapping into place is caused by the spring force of the elastic element 30. These processes are explained in more detail below.

The connecting element 12 may receive one or more tubes 14. Arms 16 may be attached to the tubes 14. Screens, holders for tools or similar items may be attached to the arms 16. Arms 16 may have joints, whereby the position of one end (remote from the tube 14) (to which screens etc. can be attached) may be flexibly adjusted.

FIG. 3 shows a schematic representation of an exemplary securing element 18 according to an embodiment. The securing element 18 is shown at the bottom in three components and at the top in the assembled state. The securing element 18 comprises two parts 28-1 and 28-2 (together 28) and an elastic element 30. The two parts 28 each comprise a bulge 32, an undercut 44, a central bore 36, and two locking elements 38. In this embodiment, the (here two) locking elements 38 each comprise a locking wall 40, bores 42, and a screw 48 with a thread 50.

The respective bulge 32 extends along the entire angle segment representing the corresponding one of the two parts 28. Thus, all bulges 32 extend once (360°) inside around the securing element 18. The bulges 32 are shaped so that they fit into the corresponding groove of the connecting element 22. The bulges 32 shown have a chamfer or sloping surface (tapered upwards in the Figure). This makes it easier to pull the securing element 18 onto the connecting element 22 by pressing the securing element apart. Preferably, there is no such sloping surface on the other side of the bulges 32 (downward in the Figure) so that the securing element 18 is not easily pressed apart when assembled (e.g., by its own weight). This surface may run straight radially outward. The bulge is preferably angular (especially with an angular right angle to the straight surface facing downwards; angular means not rounded) in order to provide better hold.

In the securing element 18 according to this embodiment, the bulges 32 result in an undercut 44. The chamfer (widening at the axial end 24 of the connecting element 22, closer to the axial end 24 than the groove 26) may fit into this undercut 44.

Here, the elastic element 30 is an elastic band that is adapted to extend around the parts 28 and press them inward (together). The parts 28 preferably have a recess 52 on the outside in which the elastic element 30 may extend to increase stability and prevent the elastic element 30 from slipping.

In a compressed state of the two parts 28, the central bore 36 is located essentially in the middle of the securing element 18. The central bore 36 may be designed with or without an internal thread.

In this embodiment, the locking elements 38 each consist of a locking wall 40, bores 42, and a screw 48 with a thread 50. The locking wall 40 may be a screw nut here. Correspondingly, a second screw nut (second locking wall) may be part of the second locking element (here shown further to the right at the top). The screw nut 40 shown is already screwed onto the thread 50 of the screw 48. For assembly, it is advantageous to screw the screw nut onto the thread 50 only after the screw 48 has been threaded through the corresponding bores 42. In this way, the screw nut 40, in conjunction with the screw 48, may hold the two parts 28-1 and 28-2 together.

The locking elements 38 and their mode of operation are explained in an alternative in more detail with the aid of FIG. 4. FIG. 4 shows a cross-section. In this embodiment, the locking elements 38 are two screws 48 and two rods 51. One respective screw 48 is guided through a bore 42 in one of the two parts 28-1 and the corresponding rod 51 is guided through a bore 42 in the other of the two parts 28-2. The screw 48 has a thread 50 on the outside. The rod 51 has a thread on the inside. The screw 48 is screwed into the rod 51 with the aid of the thread until the head of the screw 48 and the locking wall 40 of the rod 51 hold the two parts 28-1 and 28-2 firmly together. In such a held-together state, the two parts 28 cannot be accidentally pulled apart/pressed apart. The two parts 28 may comprise one screw-tube pair, two pairs, or more pairs. With two or more pairs, the two parts 28 are further stabilized against tilting relative to each other. Preferably, the pairs are arranged symmetrically on the securing element 18.

Alternatively, the locking element 38 may consist of only one screw 48. In this embodiment, at least one bore 42 of the two parts 28 has an internal thread. The screw 48 is then passed through two of the bores 42 of the two parts 28 (one respective bore 42 in each of the two parts 28) and the two parts 28 are held together by the internal thread. In the case where only one of the two (opposite) bores 42 has an internal thread, the screw 48 is first passed through the bore 42 without a thread and then through the bore 42 with a thread.

Alternatively, the locking element 38 may also be a rod and two brackets that are attached to the ends of the rod when the rod is in the bores 42.

FIG. 5 shows a schematic representation of one of the two parts 28 (here part 28-1) according to an embodiment. In this embodiment, the bulge 32 comprises three point-shaped elevations 32-1, 32-2, and 32-3. These point-shaped elevations 32-1, 32-2, and 32-3 are adapted to engage in the groove 26 of the cylindrical connecting element 22. It is also possible for the two parts 28 to have only one point-shaped elevation, two point-shaped elevations, four point-shaped elevations, or more point-shaped elevations.

FIG. 6 shows an example method 600 for attaching the securing element 18 to the cylindrical connecting element 22 according to an embodiment. Such a method comprises: (step 620) pressing apart two parts 28 of the securing element 18; (step 630) pulling the securing element 18 onto the cylindrical connecting element 22; and (step 640) pressing the two parts 28 together by means of an elastic element 30 so that a respective bulge 32 of the two parts 28 engages in a groove 26 of the cylindrical connecting element 22 in such a manner that the securing element 18 cannot be stripped off the connecting element (12, 22).

Such a method 600 may further comprise the following steps: (step 610) pulling a tube onto the cylindrical connecting element; and (step 650) if present, fastening the locking element.

1. A securing element for attachment to a cylindrical connecting element with a groove extending radially around the cylindrical connecting element, wherein the securing element comprises:
two parts, the two parts each comprising a bulge; and
an elastic element which is adapted to press the two parts together into a compressed state;
wherein the respective bulge is adapted to fit into the groove of the cylindrical connecting element and, in the compressed state, to engage in the groove of the cylindrical connecting element in such a way that the securing element cannot be stripped off the connecting element.

2. The securing element according to claim 1, wherein the respective bulge has a chamfer, and/or wherein the respective bulge is aligned with a side facing the respective other part.

3. The securing element (18) according to claim 1, wherein the securing element is adapted to be pulled at least partially over an axial end of the connecting element.

4. The securing element according to claim 1, further comprising a locking element which is adapted to fix the securing element in the compressed state, and preferably wherein the locking element (38) is at least one screw (20, 48).

5. The securing element according to claim 1, further comprising two rods, wherein the two parts each comprise two bores, wherein a respective one of the two rods is adapted to be guided through a respective bore in one of the two parts, and preferably wherein at least one of the two rods is hollow and comprises a thread which is adapted to receive the screw, and wherein each of the two rods comprises a respective locking wall which is located on an outer side of the respective one of the two rods and which is larger than a diameter of the bores of the two parts.

6. The securing element according to claim 1, wherein the respective bulges are together adapted to extend, in the compressed state, radially around the connecting element in the groove.

7. The securing element according to claim 1, which is adapted to have a central bore in the radial center in the compressed state.

8. The securing element according to claim 1, wherein the securing element further comprises:
more than two parts comprising the two parts, wherein the more than two parts each comprise a bulge.

9. A system comprising:
a cylindrical connecting element with a groove extending radially around the cylindrical connecting element; and
the securing element according to claim 1.

10. A method for attaching a securing element to a cylindrical connecting element, comprising:
pressing apart two parts of the securing element;
pulling the securing element onto the cylindrical connecting element; and
pressing together the two parts by means of an elastic element so that a respective bulge of the two parts engages in a groove of the cylindrical connecting element in such a manner that the securing element cannot be stripped off the connecting element.

11. The securing element according to claim 4, wherein the locking element is at least one screw.

12. The securing element according to claim 5, wherein at least one of the two rods is hollow and comprises a thread which is adapted to receive the screw, and wherein each of the two rods comprises a respective locking wall which is located on an outer side of the respective one of the two rods and which is larger than a diameter of the bores of the two parts.
